(19) United States
(12) Patent Application Publication
(10) Pub. No.: US 2024/0341679 A1
(43) Pub. Date: Oct. 17, 2024

(54) JOINT MEASUREMENT DEVICES, SYSTEMS, AND METHODS

(71) Applicant: Orthosensor, Inc., Dania Beach, FL (US)

(72) Inventor: Joseph DeCERECE, Fort Lauderdale, FL (US)

(73) Assignee: Howmedica Osteonics Corp., Mahwa, NJ (US)

(21) Appl. No.: 18/682,604

(22) PCT Filed: Aug. 8, 2022
(86) PCT No.: PCT/US2022/039667

§ 371 Date: Feb. 9, 2024

Related U.S. Application Data
(60) Provisional application No. 63/231,896, filed on Aug. 11, 2021.

(51) Int. Cl. A61B 5/00; A61B 5/07; A61F 2/30; A61F 2/36

(52) U.S. Cl. CPC A61B 5/4851; A61B 5/076; A61B 5/686; A61B 5/742; A61F 2/3662; A61B 2562/0261; A61F 2002/305; A61F 2002/3625

(57) ABSTRACT

A medical device used for measuring loads at joints. The device may have a stem coupled to bone, a neck, and a ball joint coupled to the neck. The ball joint may be a femoral trial head. The ball joint may be an upper and lower housing coupled together. The ball joint houses a central column that may be a part of the lower housing, a circuit board on the column, and sensors. The sensors may be radially arrange around the circuit board at equal distances from the circuit board and equal angular distances from each other. The sensors may be impacted by features on the inner surface of the upper housing so that they may together measure the force on the upper housing. The force magnitude and location at the joint may be determined from the forces measured at the sensors.

### CROSS-REFERENCE TO RELATED APPLICATIONS

This application claims the benefit of priority from U.S. Provisional Application No. 63/231,896, filed Aug. 11, 2021, which is incorporated by reference herein in its entirety

### TECHNICAL FIELD

The present disclosure relates generally to medical devices for measuring parameters at joints, among other aspects.

### BACKGROUND

The skeletal system of a mammal is subject to variations among species. Further changes can occur due to environmental factors, degradation through use, and aging. An orthopedic joint of the skeletal system typically comprises two or more bones that move in relation to one another. Movement is enabled by muscle tissue and tendons attached to the skeletal system of the joint. Ligaments hold and stabilize the one or more joint bones positionally. Cartilage is a wear surface that prevents bone-to-bone contact, distributes load, and lowers friction.

There has been substantial growth in the repair of the human skeletal system. In general, orthopedic joints have evolved using information from simulations, mechanical prototypes, and patient data that is collected and used to initiate improved designs. Similarly, the tools being used for orthopedic surgery have been refined over the years but have not changed substantially. Thus, the basic procedure for replacement of an orthopedic joint has been standardized to meet the general needs of a wide distribution of the population.

Although the tools, procedure, and artificial joint meet a general need, each replacement procedure is subject to significant variation from patient to patient. The correction of these individual variations relies on the skill of the surgeon to adapt and fit the replacement joint using the available tools to the specific circumstance. The gathering of relevant data on parameters within the joint may assist the surgeon in replacing the joint. The solution of this disclosure resolves these and other issues of the art.

### SUMMARY

In one aspect, a measurement device may include a stem configured to couple to a bone at a proximal end portion; a neck extending outward from a distal end portion of the stem; a ball joint coupled to the neck, the ball joint comprising a lower housing and an upper housing. The lower housing may include a center body with a platform at a first end of the center body; a circuit board with electronic circuitry coupled to the platform; and a plurality of sensors circumferentially arranged around the center body and spaced from the platform; and wherein the ball joint is configured to couple to a joint and to measure a load magnitude and location at the joint.

In other aspects, the measurement device may include one or more of the following features. The measurement device may further include a shim; wherein the neck fits inside the shim and the shim fits at least partially inside an opening in the lower housing. At least one battery may be within the ball joint and may be positioned around the center body. The sensors may be strain gauges. The lower housing may further comprise sensor holding features for holding the sensors at an offset height and an offset angle from the platform. The measurement device may further include lower housing snap features on the lower housing; upper housing snap features on the upper housing; wherein the lower housing snap features and the upper housing snap features are both equal in number to the number of sensors; wherein the lower housing and the upper housing are coupled together with the lower housing snap features and the upper housing snap features; and wherein the lower housing snap features and the upper housing snap features are aligned with the sensors. The sensors may be coupled to flexible circuit board portions that are coupled to the circuit board. The electronic circuitry may be connected to an external antenna outside the ball joint, and the external antenna may be configured to communicate with a remote system. The measurement device may be configured to communicate with a remote system and the remote system displays the load magnitude and location on a graphical user interface (“GUI”). The GUI may include a display of the ball joint with concentric rings, wherein the concentric rings are configured to indicate a distance of the force from the center of the upper housing.

In other aspects, a measurement device may include a stem configured to couple to a bone at a proximal end portion; a neck extending outward from a distal end portion of the stem; a shim coupled to the neck; and a ball joint coupled to the shim, the ball joint comprising a lower housing and an upper housing. The lower housing may comprise a center body with a platform at a first end of the center body; a circuit board with electronic circuitry coupled to the platform; and three sensors circumferentially arranged around the center body and spaced from the platform; and wherein the ball joint is configured to couple to a joint and to measure a load magnitude and location at the joint.

In other aspects, the measurement device may include one or more of the following features. The shim may include release arms that are accessible through gaps in the lower housing. At least one battery may be within the ball joint positioned on the side of the column. The measurement device may further include lower housing snap features on the lower housing; upper housing snap features on the upper housing; wherein the lower housing snap features and the upper housing snap features are both equal in number to the number of sensors; wherein the lower housing and the upper housing are coupled together with the lower housing snap features and the upper housing snap features; and wherein the lower housing snap features and the upper housing snap features are aligned with the sensors. The measurement device may be configured to communicate with a remote system that displays the load magnitude and location on a GUI in real-time, and the GUI includes a display of the ball joint with concentric rings that may indicate a distance of the force from the center of the upper housing.

In other aspects, a measurement device may include a stem configured to couple to a bone at a proximal end portion; a neck extending outward from a distal end portion of the stem; a shim coupled to the neck; a ball joint coupled to the shim, the ball joint comprising a lower housing and an upper housing; wherein the lower housing comprises: a center body with a platform at a first end of the center body; a circuit board with electronic circuitry coupled to the platform; and three sensors circumferentially arranged around the center body; wherein the three sensors are at an offset height and an offset angle from the platform. The neck fits inside the shim and the shim fits at least partially inside an opening in the lower housing; and the ball joint is configured to couple to a joint and to measure a load magnitude and location at the joint.

In other aspects, the measurement device may include one or more of the following features. The sensors may be coupled to flexible circuit board portions that are coupled to the circuit board. The lower housing may further comprise sensor holding features for holding the sensors at the offset height and angle. The measurement device may further comprise: lower housing snap features on the lower housing; upper housing snap features on the upper housing; wherein the lower housing snap features and the upper housing snap features are both equal in number to the number of sensors; wherein the lower housing and the upper housing are coupled together with the lower housing snap features and the upper housing snap features; and wherein the lower housing snap features and the upper housing snap features are aligned with the sensors. The measurement device may be configured to communicate with a remote system and the remote system may display the load magnitude and location on a GUI in real-time, and the GUI may include a display of the ball joint with concentric rings that may indicate a distance of the force from the center of the upper housing.

### BRIEF DESCRIPTION OF THE DRAWINGS

FIG. 1 shows a perspective view of a medical device system, according to aspects of this disclosure;

FIG. 2 shows a perspective view of an implantable medical device positioned within a portion of a patient's musculoskeletal system, according to aspects of this disclosure;

FIG. 3 shows a cross section of a ball joint of the implantable medical device of FIG. 2, according to aspects of this disclosure;

FIG. 4 shows an exploded view of the ball joint of the implantable medical device of FIG. 2, according to aspects of this disclosure;

FIG. 5 shows a perspective view of a shim that may be a component of the medical device of FIG. 2, according to aspects of this disclosure;

FIG. 6 shows a bottom view of a lower housing of the implantable medical device of FIG. 2, according to aspects of this disclosure;

FIG. 7 shows a perspective view of the lower housing of the implantable medical device of FIG. 2, according to aspects of this disclosure;

FIG. 8 shows a top view of a circuit board assembly, according to aspects of this disclosure;

FIG. 9 shows a perspective view of the lower housing of implantable medical device of FIG. 2 with the circuit board assembly of FIG. 8 positioned within the lower housing, according to aspects of this disclosure;

FIG. 10 shows a perspective view of an upper housing of the implantable medical device of FIG. 2, according to aspects of this disclosure;

FIG. 11 shows a view of a portion of the bottom of the upper housing of FIG. 10, according to aspects of this disclosure;

FIG. 12 shows the remote system and two example graphical user interfaces (GUI) that may be used to display information received by the remote system from the implantable medical device of FIG. 1, according to aspects of this disclosure;

FIG. 13 shows an alternate target measuring area of the device of FIG. 1; and

FIG. 14 shows a flowchart of the data transmission from the medical device to the remote system of FIG. 1.

### DETAILED DESCRIPTION

Both the foregoing general description and the following detailed description are exemplary and explanatory only and are not restrictive of the features, as claimed. As used herein, the terms “comprises,” “comprising,” “having,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. Moreover, in this disclosure, relative terms, such as, for example, “about,” substantially,” and “approximately” are used to indicate a possible variation of +10% in the stated value.

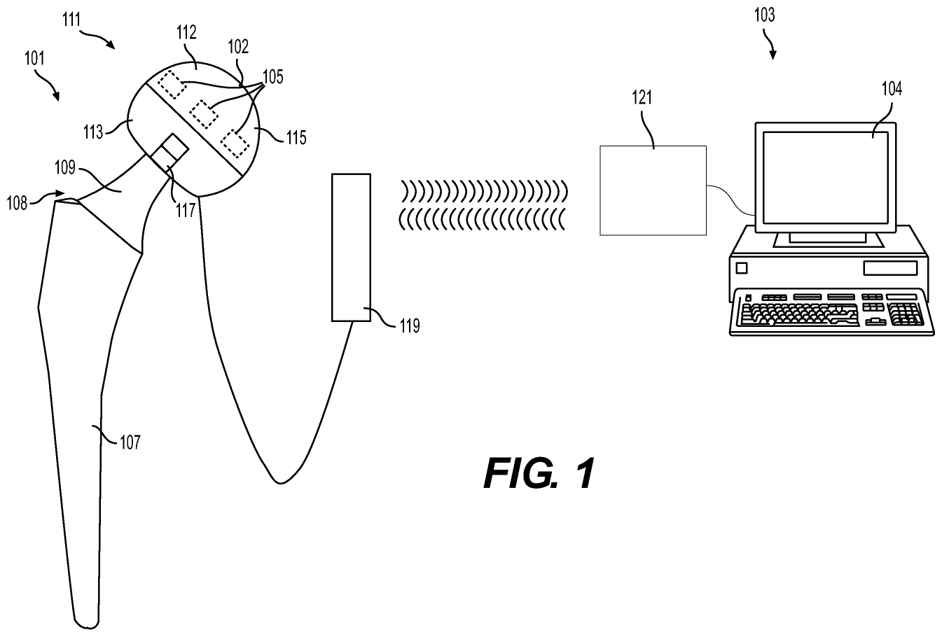

FIG. 1 illustrates a medical device 101 that may be used to measure parameters of a patient's muscular-skeletal system. For example, medical device 101 may measure one or more parameters, and send those parameters to a remote system 103 where the parameters may be processed and/or displayed. The medical device 101 may include multiple sensors 105 that are configured to measure forces applied to a joint or other parameters. In some examples, the magnitude and location of a force may be determined based on combining the measured forces at each individual sensor 105. The medical device 101 may comprise a stem 107, a neck 109, and a ball joint 111. The stem 107 may be coupled to a bone on a distal portion of the stem 107. The neck 109 may be the proximal portion 108 of the stem 107 where the ball joint 111 is coupled. The ball joint 111 may comprise a lower housing 113 coupled to an upper housing 115. A center point 102 of the ball joint 111 may be in the center of the ball joint relative to the neck 109. The ball joint 111 may house electronic circuitry 401 and sensors 105. In one embodiment, the lower housing 113 has a recessed portion 601 on its bottom surface 606 that may at least partially receive a shim 117. The shim 117 may fit around the neck 109, coupling the ball joint 111 to the neck 109. Alternatively, the neck 109 may be directly coupled to the ball joint 111 without the shim 117.

The medical device 101 gathers data using the sensors 105 housed within the ball joint 111. The data is then sent to a transmitter 119. As shown, the transmitter 119 is an external antenna 119. However the transmitter 119 may be internal and contained within medical device 101 (e.g. within a stem 107, a neck 109, and a ball joint 111), or external to medical device 101. The transmitter 119 may be any data transmission device known in the art and may use any known data transmission protocol. After electronically transmitting the sensor data to the transmitter 119, the data is then transmitted to a remote system 103. The transmission may be wireless, such as over Wi-Fi, Bluetooth, the internet, or other common wireless communication protocols. Alternatively, the transmission may be over a wired connection with the remote system. A receiver 121 receives the data transmission from the transmitter 119. The receiver 121 may be external or internal to the remote system 103. The data may then be processed and displayed on the remote system 103, such as on electronic display 104, as described further below.

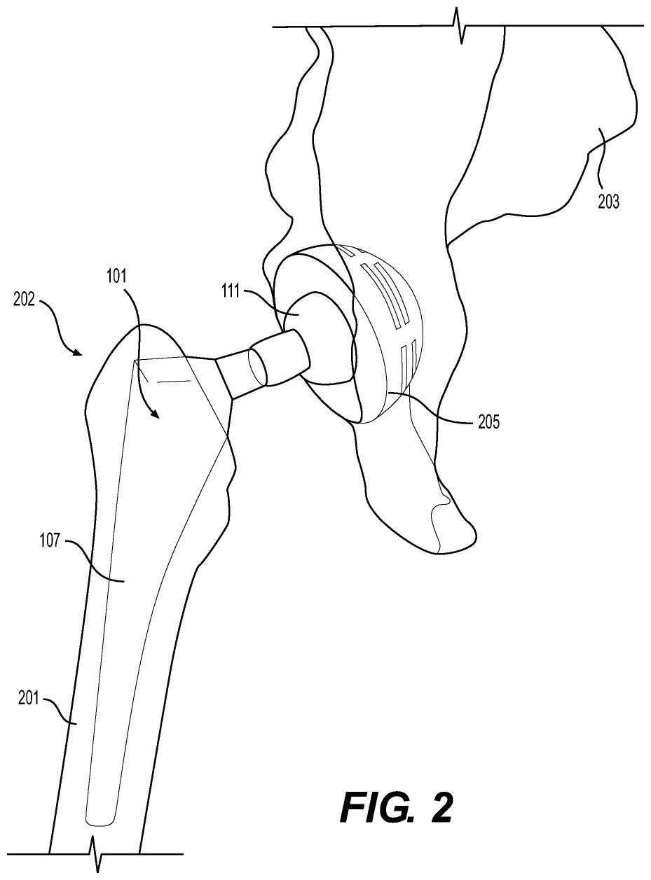

FIG. 2 shows the medical device 101 coupled to a patient's skeletal system. In one embodiment, the stem 107 may be a femoral stem configured to couple to a femur 201 of a patient and the ball joint 111 is configured to couple to a patient's hip bone 203. The stem 107 may be configured to extend longitudinally through femur 201 and be inserted into femur 201 at a proximal end portion 202 of femur 201. The ball joint 111 is coupled to a prepared area 205 on the hip bone 203. The prepared area 205 of the patient's hip 203 bone may be prepared to receive the ball joint 111 by cutting bone and/or coupling a receiving prosthetic joint cup 205 to the hip bone 203 that may receive the ball joint 111. The ball joint 111 may be a femoral trial head used during surgery to measure the magnitude, location, and vector of forces placed on the ball joint 111. The medical device 101 may be used by medical personnel as a temporary measurement device during a surgery, and may be removed during surgery prior to implanting a permanent implant. For example, a surgeon may use the ball joint 111 as a femoral trial head to determine the force a patient's muscular-skeletal system imparts on a hip joint at the leg's current position and bone alignment. In some examples, medical device 101 may be a permanent implant device that remains within the body of the patient for an extended period of time post-surgery. The surgeon may then make informed decisions, such as decisions on correct positioning, balancing loads on the joint, and size of a prosthesis.

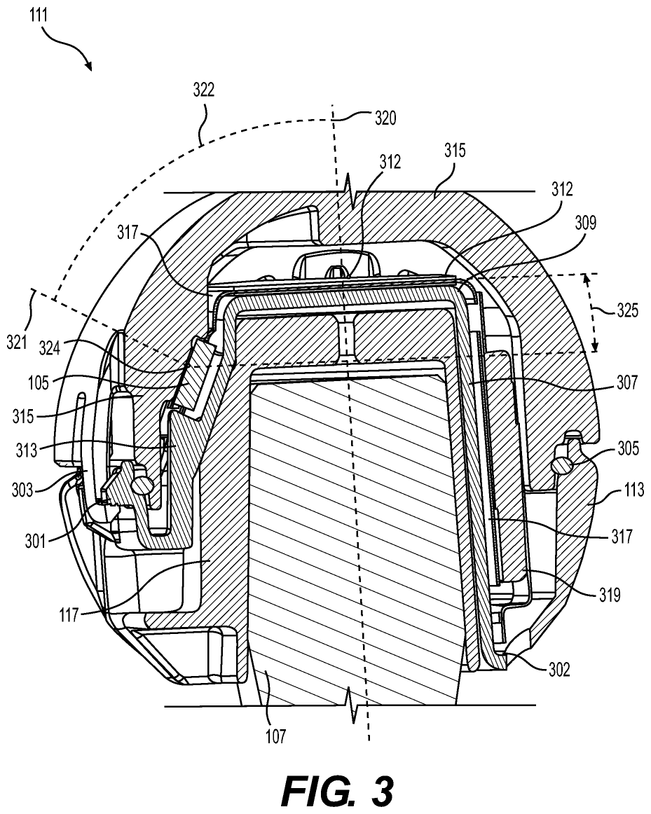

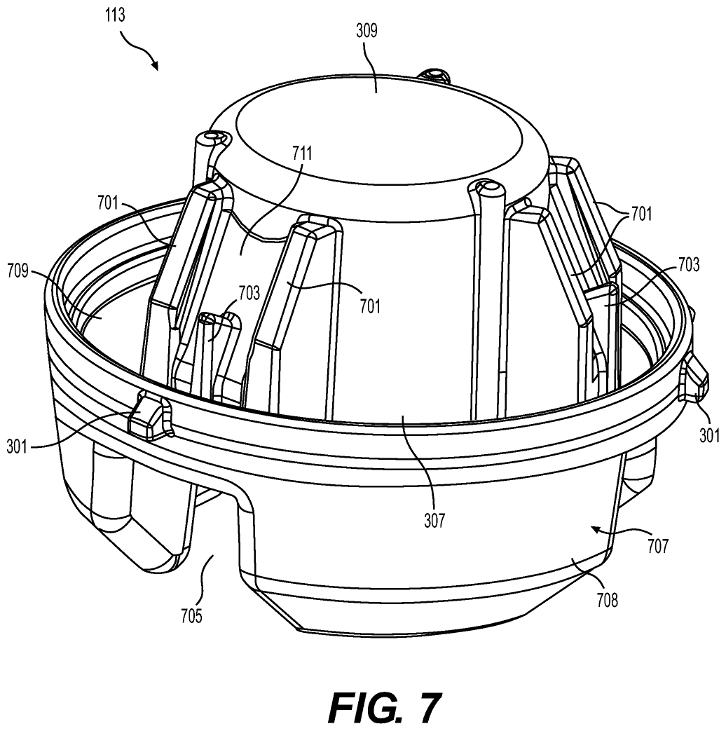

FIG. 3 shows a cross section of the ball joint 111, shim 117, and neck 109. As discussed above, the lower housing 113 couples to the upper housing 115 to form the ball joint 111. The lower housing 113 may have a lower housing snap feature 301 on the lower housing outer surface 114 and the upper housing 115 may have an upper housing snap feature 303 on the upper housing outer surface 116. In one embodiment, the lower housing snap feature 301 may be the inserting portion (or male portion) and the upper housing snap feature 303 may be the receiving portion (or female portion) which receives the lower housing snap feature 301. An O-ring 305 may be situated between the lower housing 113 and upper housing 115. The lower housing 113 may contain a central column 307, and the central column may be raised relative to the base 302 of the lower housing 113 interior surface 304. The top surface 309 of the column 307 may form a platform 309. A circuit board 311, such as a printed circuit board (“PCB”), may be situated on the platform 309. The lower housing 113 may have sensor holding features 313 for holding sensors 105 at a separate height and angle from the circuit board 311. The sensor holding features 313 may be support structures that extend radially from the column surface 308 of column 307. The sensor holding features may extend from the lower housing base 302 to the platform 309. The sensor holding features may include two end support structures 701 and a middle support structure 703 (see FIG. 7). The upper housing 115 may have sensor impacting features 315 on the interior surface 1103 of the upper housing 115. The sensor impacting features 315 may be flat surfaces normal to angle 322. The sensor impacting features 315 may protrude radially from interior surface 1103 of the upper housing 115 toward column 307 and sensors 105. The sensor impacting features 315 may be designed to transfer the force on the outside of the upper housing 115 to the sensors 105 so that the sensors 105 may measure the total force on the upper housing 115 together. The sensor impacting features 315 may be angled normal to the surface of the sensors 105.

Sensors 105 within the ball joint 111 may be attached to portions of circuit board 311. In some examples, the sensors 105 may be load or force detecting sensors. For example, the sensors may be strain gauges and/or capacitive force sensors. The sensors 105 may be coupled to flexible circuit board portions 317 that may be positioned along the column surface 308 of column 307. The sensors 105 may be placed radially around the central column 307 or platform 309, and each sensor 105 may be evenly spaced between two adjacent sensors 105. The sensors 105 may be placed at equal distances from the center of the platform 309. For example, with three sensors 105, there may be 120 degrees between each sensor while all three sensors form a circular shape around the center of the platform 309. The center point 324 of sensors 105 may have an offset height 325 from the circuit board center point 312 such that they are relatively lower or higher than the circuit board 311. Each sensor surface 106 may be normal to an angle 322 from the circuit board 311 such that the sensors 105 may be from 0 degrees to 90 degrees offset from the circuit board 311 top surface. In one embodiment, three strain gauge sensors 105 are coupled to flexible PCB 317, and each strain gauge sensor 105 is spaced radially around the PCB 317 on the platform 309. The three sensors 105 may be at equal distances from a center axis 320 that extends from the center of neck 109 through the circuit board center point 312 and through the center 102 of ball joint 111. The three sensors 105 may be at an offset height 325 from the circuit board 311. A sensor axis 321 that extends normal to the sensor surfaces 106 of each sensor 105 may be at an angle 322. The sensor may share equal acute angles 322 from the circuit board 311 such that the sensor surfaces 106 of the sensors 105 are facing the interior surface 1103 of the upper housing 115. The three sensors 105 may each be impacted by, or in contact with, one of three sensor impacting features 315 of the upper housing 115. The interaction between the three sensors 105 and the three sensor impacting features 315 may allow the magnitude and location of the load on the upper housing 115 to be determined, for example by measuring the load applied to upper housing 115.

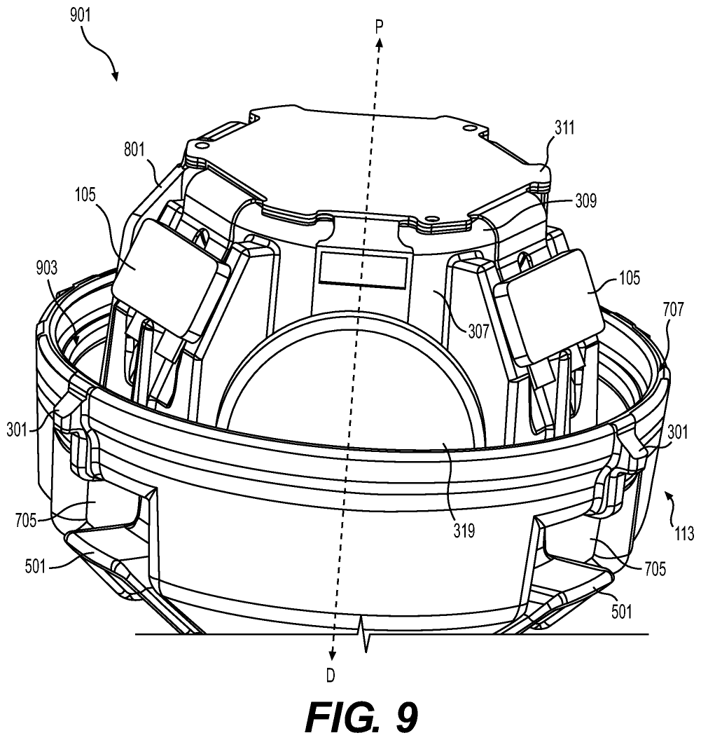

At least one battery 319 or other power source may be housed within the ball joint 111 or other portion of medical device 101. The at least one battery 319 may be coupled to a flexible circuit board portion 317 that is attached to the circuit board 311 on the platform 309. In some examples, batteries 319 may be positioned in spaces between the column 307 and the inner surface of the ball joint 111. The batteries 319 may be relatively distal to the circuit board 311 on the platform 309 (see FIG. 9). The batteries 319 may be positioned at or approximately 90 degrees from the center axis 320. The batteries 319 provide a power source for the sensors 105, electronic components 401, programming port 803, controller 805, antenna plug 807, and any other known electronic circuitry that may be within the ball joint 111. The batteries 319 may also provide power to an external or internal transmitter 119, such as an antenna.

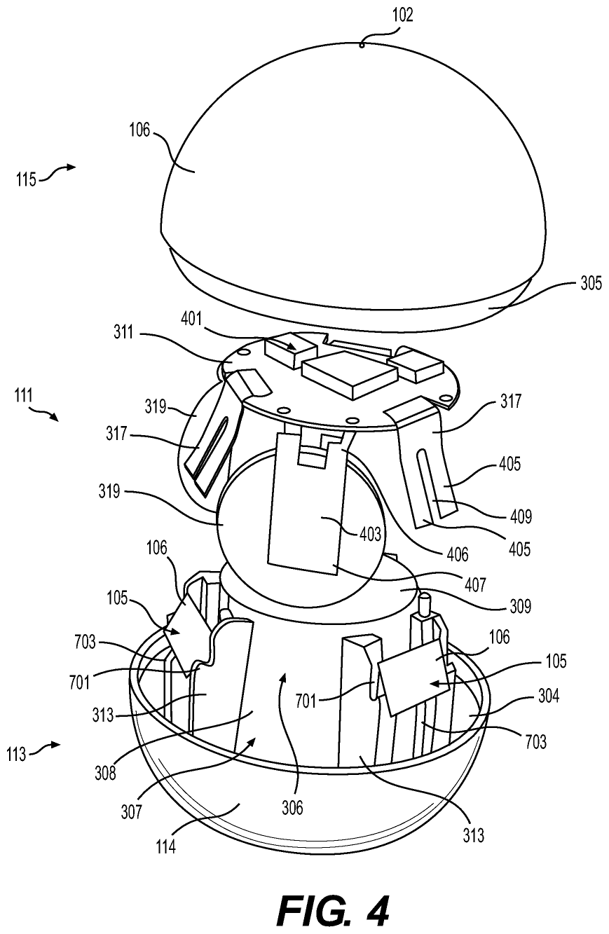

FIG. 4 is an exploded view of the ball joint 111. Lower housing 113 may have a raised platform 309 or column 307 at the center of the lower housing interior surface 304. The column 307 may be cylindrical. Lower housing 113 may have sensor holding features 313 to hold the sensors 105 at the desired position, height, and angle. The sensor holding features 313 may extend radially from the column surface 308. As described above, the sensor holding features 313 may include two end support structures 701 and a middle support structure 703. The end support structures 701 may be flat surfaces normal to angle 322 so that sensor surface 106 is also normal to angle 322. There may be a sensor holding recess 711 between the end support structures 701. The middle support structure 703 may be a flat surface normal to the end support structures 701 that extend radially from column 307 at angle 322. The sensor holding features 313 may be configured such that when sensors 105 lay across end support structures 701 at the correct angle 322, the middle support structure 703 supports the weight of sensors 105. In one embodiment, the sensors 105 may be held in place by structural elements of the sensor holding features 313, such as middle support structure 703, and are not directly mounted to the sensor holding features 313 by, for example, an adhesive or other coupling means. In other examples, sensors 105 are glued or otherwise adhered to sensor holding features 313. As described further below, the sensors 105 held in place by the sensor holding features 313 may be mounted by a mounting means to flexible circuit board portions 317. There may be an equal number of sensor holding features 313 and sensors 105, for example 3, 4, 5, 6, 7, or any other suitable number. In one embodiment, three sensors 105 and three sensor holding features 313 are equally spaced around the column 307.

In one embodiment, the column 307 may have a flat circular platform 309 at the top portion or end 306 of the column 307. A circuit board 311, such as a PCB, may be positioned on the platform 309 of the column 307. The circuit board 311 may be the same or similar shape and size as the platform 309 of the column 307, such as substantially circular. The circuit board 311 may have electronic components 401 mounted or printed on the top surface of the circuit board 311. Circuit board connector portions 403 may be circuit board portions coupled to the circuit board 311 at a first end 406 and may be coupled to another component of the ball joint 111 on the a second end 407. These circuit board connector portions 403 may be circuit board pieces that extend from the circuit board 311 towards the lower housing base 302 to hold components in a space 903 between the column 307 and interior surface 304 of the lower housing of the ball joint 111. The circuit board connector portions 403 may each be separate circuit board pieces that are coupled to the circuit board 311. In other examples, the circuit board connector portions 403 may be the same piece of circuit board as, or integral with, the circuit board 311. For example, circuit board 311 may be cut to create circuit board connector portions 403. Circuit board connector portions 403 may be flexible circuit board portions 317. The flexible circuit board portions 317 may be two PCB arms 405 formed with a relief cut space 409 between the two arms 405. This allows each of the two arms 405 of the flexible circuit board portions 317 to move independently of one another, so that any bending of the sensor 105 due to an impacting force will not harm the circuit board 311 or flexible circuit board portions 317. The sensors 105 may be mounted on the flexible circuit board connector portions 403 using any known mounting means. Power sources, such as batteries 319, and/or other components may also be mounted to circuit board connector portions 403. In one embodiment, two batteries 319 coupled to circuit board connector portions 403 are each positioned between sensor holding features 313 in the space 903 between the column 307 and the inner surface of the ball joint 111.

The lower housing 113 may couple to the upper housing 115 to seal the ball joint 111. In one embodiment, the lower housing 113 and the upper housing 113 couple together using snap features 301 and 303 (see FIG. 3). Lower housing snap feature 301 may extend radially outward, relative to a central longitudinal axis extending in the proximal-distal direction, from the lower housing 113. Upper housing snap feature 303 may extend from the upper housing outer surface 1001 towards the lower housing 113. Upper housing snap feature 303 may include a lumen 1003 that passes fully through upper housing snap feature 303. The lower housing snap 301 feature may be angled or otherwise configured so that when the lower housing snap feature 301 passes through lumen 1003, the lower housing snap feature 301 may not be removed from lumen 1003 without an external force, coupling snap features 301 and 303 together. An O-ring 305 may compress between the upper housing 115 and a lip 1005 or similar structural feature on the lower housing 113 to seal the ball joint 111. Upper housing 115 receives force applied from a musculoskeletal system of a patient, which is transmitted to the sensors 105 positioned in the lower housing 113. Upper housing 115 may include structural elements, such as internal ribbing 1105, to give upper housing 115 support to receive forces while in use and may help prevent damage or breaking of upper housing 115 due to excessive force. Similarly, lower housing 113 may have support structures on its internal and/or external surface to support lower housing 113 (not shown).

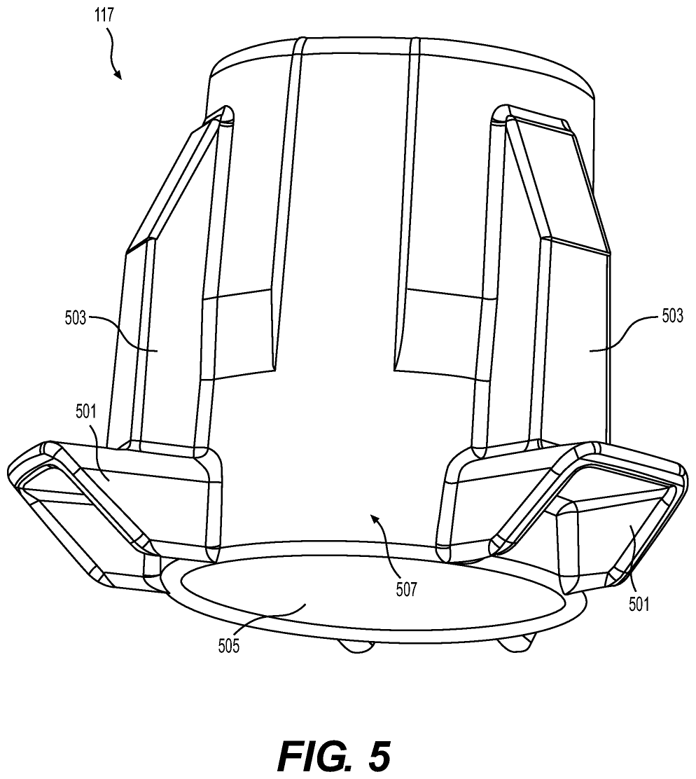

FIG. 5 shows the shim 117 separately from medical device 101. The shim 117 may fit wholly or partially inside the lower housing 113 of the ball joint 111. The neck 109 may fit inside a shim lumen 505 of the shim 117. The shim lumen is an opening in the shim configured to receive the neck 109. In one embodiment, the shim 117 includes release arms 501 that may be used to remove the shim 117 from the ball joint 111 or to remove the shim 117 or ball joint-shim assembly from the neck 109. The release arms 501 may extend radially outwards from the column 307 in a “U” shape. The release arms 501 may be angled radially inward at the distal end 507 of the shim 117. The release arms 501 are accessible in gaps 705 in the lower housing 113, as described further below. The shim 117 may have shim support structures 503 that may fit into shim receiving portions 603 of the lower housing 113. The shim support structures 503 may assist in coupling the shim 117 to the lower housing 113, and may help prevent rotation of shim 117 relative to lower housing 113. The shim 117 may act as a load support for the ball joint 111. One advantage of this type of load support (e.g. the use of shim 117 in medical device 101) is that when a load is released from the upper housing 115 of the ball joint 111, the shim 117 may provide a force, for example a spring force, so that there is no residual load on the sensors 105 from compression and/or movement of the ball joint 111 or medical device 101.

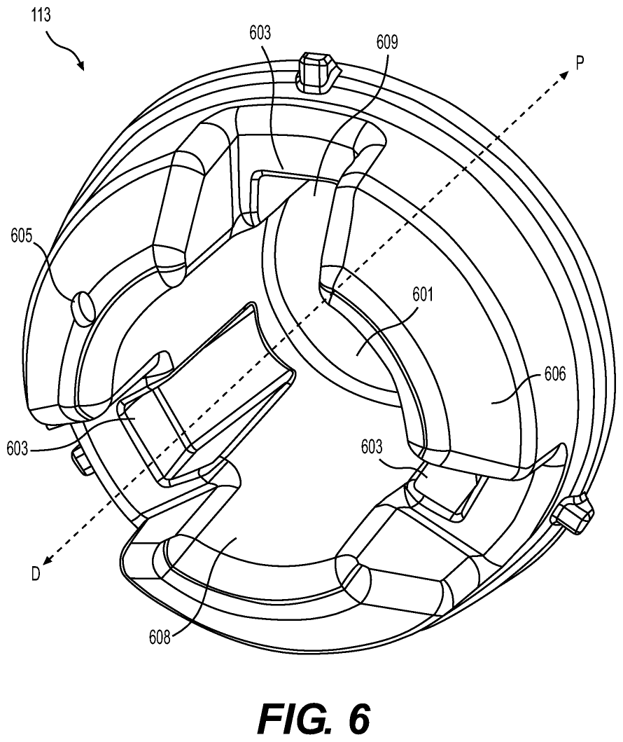

FIG. 6 shows a bottom view of the lower housing 113 of the ball joint 111. The lower housing 113 has a recessed portion 601 configured for receiving the shim 117. The recessed portion 601 may extend through an interior portion of the central column 307 and may be cylindrical in shape. The recessed portion 601 may be formed by a curved interior wall 608 of column 307 and an end surface 609 at a proximal portion of the lower housing 113 facing a distal direction. Shim receiving portions 603 may be formed from recessed extending proximally from a distal end of column 10, and may be evenly spaced circumferentially around the column. Alternatively, the lower housing 113 recessed portion 601 may receive the neck 109 directly without a shim 117. In this case, the recessed portion 601 may be coupled to the neck 109 using any method known in the art. In one embodiment, lower housing 113 has a pass-through 605 in the lower housing bottom surface 606 so that a wire or other connection may extend from within the interior portion of the housing formed by the upper housing and the lower housing to an external device, such as an external antenna or other transmitter 119. The antenna 119 may be any type of antenna known in the art. Alternatively, the antenna 119 may be inside the ball joint 111.

FIG. 7 shows a perspective view of the lower housing 113 of the ball joint 111. In one embodiment, that lower housing 113 has lower housing snap features 301 on the external surface 708 of the lower housing wall 707. The lower housing snap features 301 that may be used for coupling the lower housing 113 to the upper housing 115. There may be three lower housing snap features 301 positioned radially around the lower housing wall 707 that protrude outwardly from the lower housing wall 707. The lower housing snap features 301 may each be in equal radial positions as a middle support structure 703 of the sensor holding features 313, described below. The lower housing snap features 301 may be angled so that when received by upper housing snap features 303, the lower housing snap features couple the lower housing 113 to the upper housing 115. Alternatively, the lower housing 113 may have other known coupling features to couple to the upper housing 115. The lower housing wall 707 includes gaps 705 where the shim release arms 501 may be accessed with a finger or tool. In one embodiment, the lower housing wall 707 may also include ribbing or other support structures (not shown) on the external surface 708, internal surface 709, or both the external surface 708 and internal surface 709. The lower housing 113 may include a raised central column 307 with a flat surface platform 309. The column 307 may extend higher than the lower housing wall 707 that makes up portions of the exterior surface 112 of the ball joint 111.

The lower housing 113 includes sensor holding features 313 for holding sensors 105 at a desired position, height, and angle. In one embodiment, the sensor holding features 313 may include two end support structures 701 and a middle support structure 703 that each extend radially outward from the center column 307. The end support structures 701 and the middle support structure 703 may hold the sensors 105 at the desired angle 322 and offset height 325 (see FIG. 3). The two end support structures 701 have the same surface angle 322 and are spaced from each other so that a sensor 105 may lay across the sensor holding recess 711 between the end support structures 701 with surface 106 of the sensor 105 parallel to the end support structures 701. The middle support structure 703 may extend further radially-outward from the central column 307 than the end support structures 701 so that the sensor 105 may rest on top of the middle support structure 703. The end support structures 701 and middle support structures 703 may be angled and positioned so that the sensors 105 that rest on them are at the desired location, height, and angle. The sensors 105 may be mounted to flexible circuit board portions 317 and may not be directly coupled to the sensor holding features 313. However, the sensor holding features 313 may support the sensors 105 when under load, ensuring that the sensors 105 are at the desired location and angle 322 so that the sensors 105 may properly measure the load. The sensor holding features 313 may keep the sensor 105 held at sensor angle 322 normal to the sensor impacting features 315 of upper housing 115. The sensors 105 may be positioned between the column 307 and upper housing 115 interior surface 1103 at the offset height 325, so that the sensors 105 are positioned to maximize the measuring area on the upper housing exterior surface 1001.

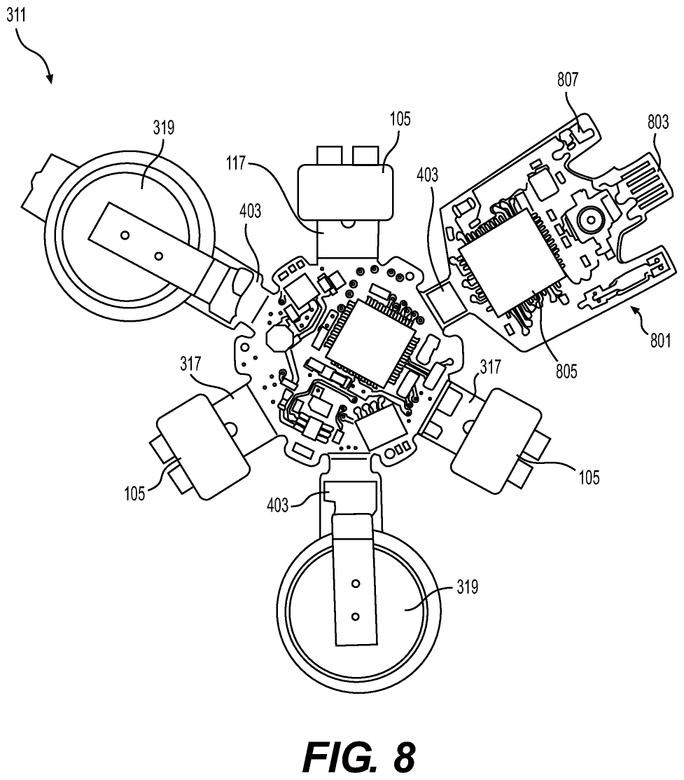

FIG. 8 shows a circuit board assembly that may be positioned within the ball joint 111. The circuit board 311 may be a PCB. The sensors 105 may be coupled, either directly or indirectly, to flexible circuit board portions 317. At least one battery 319 may be coupled to circuit board connecting portions 403 that are circumferentially arranged around the central circuit board 311. The batteries 319 and circuit board connecting portions 403 may be positioned between two sensors 105 and their associated flexible circuit board portions 317. An electronic components portion 801 may be coupled to a circuit board connecting portion 403 and may be electrically connected to the batteries 319. The electronic components portion 801 may house electronic components such as a programming port 803, a controller 805, and an antenna plug 807. The antenna plug 807 may be where an electrical wire connecting an external transmitter 119 is connected. Alternatively, the antenna plug 807 may be an internal antenna or other transmitter 119 incorporated in electronic components portion 801 or another portion of circuit board 311. The circuit board 311 may be positioned on platform 309 of the central column 307. The sensors 105, batteries 319, electronic components portion 801, and associated connecting circuit boards 403 may be positioned circumferentially around central circuit board 311 with equal angular distance between them. The sensors 105, batteries 319, electronic components portion 801, and associated connecting circuit boards 403 may also be positioned relatively in a distal direction from the central circuit board 311 (see FIG. 9) in space between the ball joint 111 inner surface and the central column 307.

FIG. 9 illustrates a lower housing sensor assembly 901 with the circuit board 311 positioned on the platform 309 of the central column 307. The release arms 501 may be accessible, or exposed to the exterior of ball joint 111 to be accessed by a user, in gaps 705 in the lower housing 113. The circuit board 311 may be coupled to the platform 309 of the central column 307 with any known coupling means. Three sensors 105 may be positioned circumferentially around the circuit board 311 on three sensor holding features 313 that are approximately 120 degrees apart, and each of the three sensors 105 may be spaced an equal distance from the center of the circuit board 311. The sensors 105 are coupled to flexible circuit board portions 317. Two batteries 319 may be coupled to connecting circuit board portions 403. Each battery 319 may be positioned between two sensors 105 in the space between the central column 307 and the lower housing wall 707. The electronic component portion 801 may be positioned circumferentially between two sensors 105 and radially in the space 903 between the lower housing interior surface 304 of the lower housing wall 707 and the column 307. The sensors 105 may also be positioned in space 903, radially inward from the lower housing wall 707.

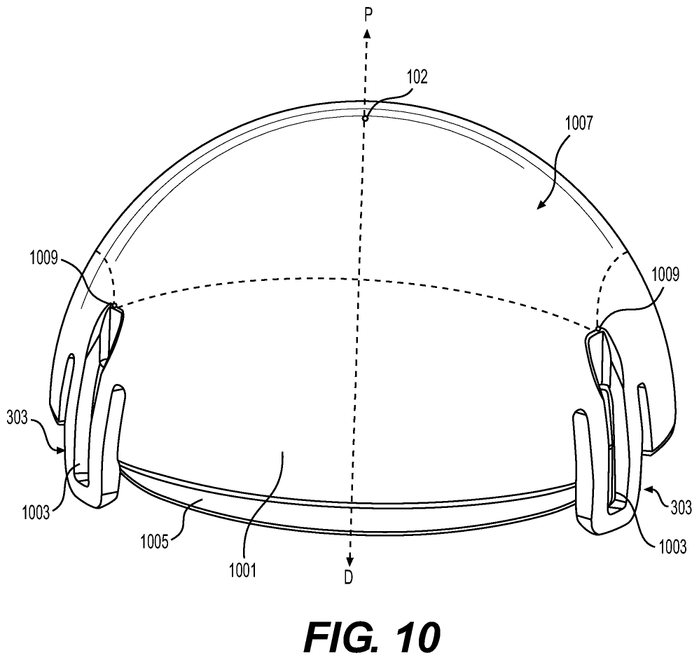

FIG. 10 shows the upper housing 115 of the ball joint 111. The upper housing outer surface 1001 may be a curved, hemispherical surface. In one embodiment, the upper housing 115 may have upper housing snap features 303. Each upper housing snap feature 303 may have a lumen 1003 to receive a lower housing snap feature 301 and lock the lower housing snap feature in place 301. The lumen 1003 may be shaped as a longitudinal axis transverse to the opposite-facing wall of the upper housing 115. The lumen 1003 may extend distally relative to the lip 1005 of the upper housing 115. The lumen 1003 may pass-through the upper housing snap feature in whole or in part. The coupling of the upper housing snap feature 303 and the lower housing snap feature 301 may secure the lower housing 113 to the upper housing 115. Alternatively, one skilled in the art would know that the upper and lower housing 113 may be coupled using other coupling means, such as adhesive or other mechanical coupling means known in the art, and be modified to include any necessary structures.

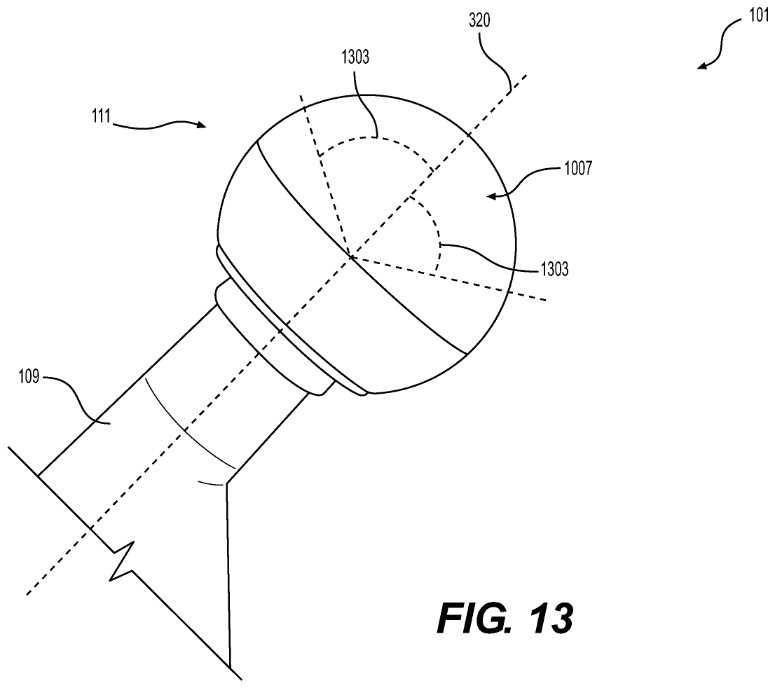

The arrangement of sensor assembly 901 and its relative interaction with upper housing 115 may allow medical device 101 to accurately measure load applied to a target area 1007 on the upper housing 115 of the ball joint 111. The size and shape of the target area 1007 depends on the positioning and angle of the sensors 105, and medical device 101 may also measure load applied to portions outside of target area 1007. The target area 1007 may be the area of the upper housing 115 that is positioned relatively proximal to the sensors 105. The target area 1007 may be a spherical polygon on the surface of the ball joint 111 where the vertices of the polygon are points 1009 on the upper housing 115 normal to the center of the sensors. Alternatively, FIG. 13 shows that target area 1007 may be a circular area of the upper housing 115 within an angle 1303 of 60 to 65 degrees of a center axis 320 that extends from the center of the neck 109 through the ball joint 111. Other methods may be used to determine the load outside the target area 1007 using sensors 105. When the snap features 301 303 are aligned with the load sensors 105, they may create a measurable counterforce on the two opposing sensors 105. An algorithm or correction factor may then be used to calculate the load outside the target area 1007 based on this measured counterforce.

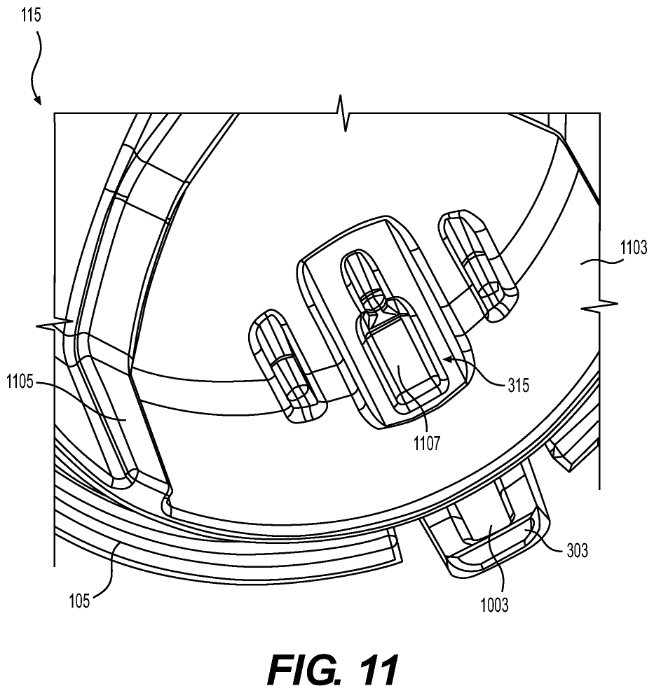

FIG. 11 shows the interior surface 1103 of the upper housing 115. The interior surface 1103 may include sensor impacting features 315. The sensor impacting features 315 may be configured to impact the sensors 105 normal to the sensor surface 106. The portions 1107 of sensor impacting features 315 where the sensor impacting features 315 are in contact with or abut the sensors 105 may be the vertices 1009 of the spherical polygon that defines the target area 1007. The interior surface 1103 of the upper housing 115 may include structural ribbing 1105 for stability and strength. The upper housing 115 may have a lip 1005 where it couples to the lower housing 113. The upper housing snap feature 303 may extend towards the neck, distal to lip 1005 so that upper housing snap feature 303 may couple to the lower housing snap feature 301.

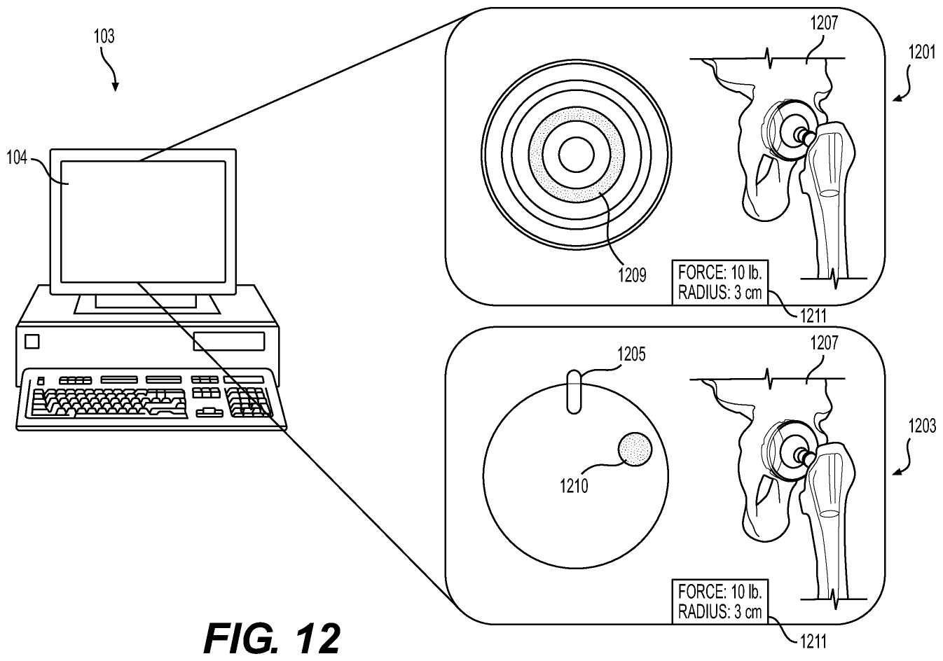

FIG. 12 shows the remote system 103 with two exemplary graphical user interfaces (“GUI”) 1201, 1203 that may be electronically displayed and used with medical device 101. The GUI 1201, 1203 may be displayed on the remote system 103, such as on an electronic display screen 104 of remote system 103. The ring GUI 1201 shows one example of a GUI where the ball joint 111 is displayed with concentric rings dividing portions of ball joint 111 to show location of the applied load on the ball joint 111 relative to the center 102 of the ball joint aligned with the stem 107. The ring 1209 that is highlighted may represent the position of the force as a radial distance from the center 102 of the ball joint 111. The ring 1209 may change color based on the magnitude of force on the ball joint 111. Different colors may be specified to represent a specific force or range of applied force. The number of rings, the distances from the center 102 of the ball joint 111 they represent, the colors, and other similar displayed information may be customizable. The ring GUI 1201 may be preferable to a surgeon over GUIs indicating the exact location of the force, and may help surgeons orient the applied force to the position of the stem 107 or neck 109 of medical device 101. The ball joint 111 may not have a specified direction to be inserted into a joint cup 205, so the exact location of the force relative to the center 102 of the ball joint 111 on a GUI may not be able to be determined without first calibrating or otherwise inputting the orientation of the ball joint 111 relative to the joint cup 205.

Alternatively, position GUI 1203 may show the exact position of the force on the ball joint 111 as a circle 1210 or otherwise highlighted small area. Ball joint 111 may be inserted into the joint cup 205 with a specified direction. For example, the ball joint 111 may have a marker 1205 indicating the desired positioning of the ball joint 111 relative to the joint cup 205. The marker 1205 may be displayed on position GUI 1203. The marker 1205 may also be indicated on the ball joint 111 and/or joint cup 205. Alternatively, the relative direction of ball joint 111 may be manually input or calibrated via controller 805 to determine the relative positioning for the position GUI 1203.

The positioning of one or more bones 201, 2013 and medical device 101 may also be displayed as part of the GUI, as shown by the positioning element 1207. The positioning of bones 201, 203 and medical device 101 may be displayed in real-time. While the bones 201, 203 and relative positioning of the medical device 101 is shown, other parts of the muscular-skeletal system may be displayed at the positioning element 1207. For example, the patient's limb, such as a leg, and the limb's relative positioning to the medical device 101 or to the operating surface may be displayed. The GUI may additionally display the force magnitude and location as a number alone or in combination with the example GUI of FIG. 12, as shown by text box 1211. The GUI may display any information that may be useful to the surgeon, such as information from additional sensors, orientation angles, alignment information, and patient information.

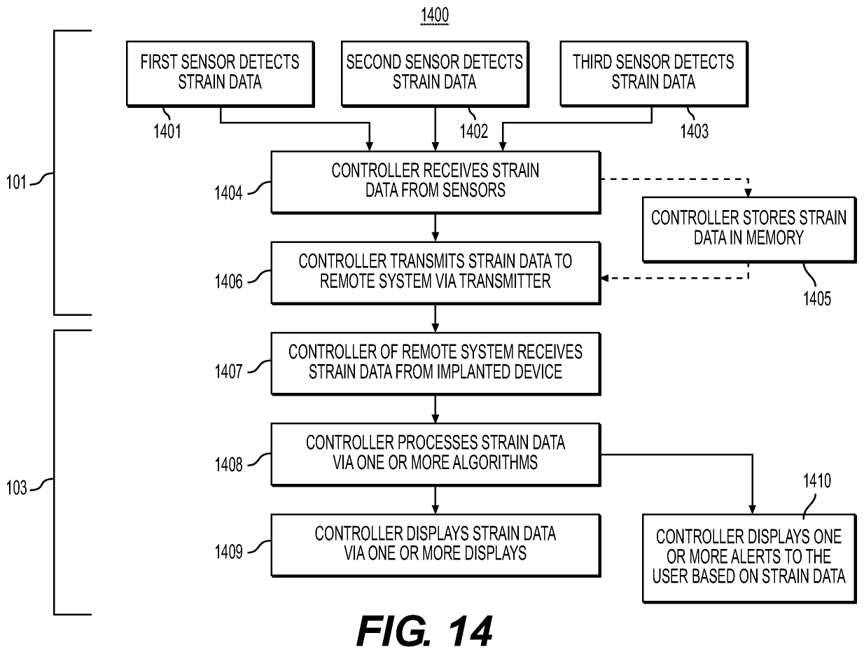

FIG. 14 shows a flowchart 1400 of the data being gathered from the sensors 105 and transmitted to the remote system 103. In one embodiment, at steps 1401-1403. within the medical device 101 the strain is detected by the sensors 105. At step 1404, the medical device controller 805 may receive the strain data from the sensors 105. Optionally, at step 1405, the controller may store strain data in memory. At step 1406, the controller 805 may transmit the strain data to remote system 103 via transmitter 119. The transmitter 119 may be an external antenna. The antenna may be any type of antenna capable of wireless communication. Alternatively, the transmitter 119 may be inside the medical device 101. At step 1407, a controller of the remote system 103 receives strain data from the medical device 101. At step 1408, the controller processes strain data via one or more algorithm. Then, at step 1409, the controller displays strain data via one or more displays, and simultaneously at step 1410 the controller may display one or more alerts to the user based on the strain data.

In some examples, a method of using the medical device 101 as a trial insert may be performed intra-operatively by a surgeon in order to make informed decisions on correct positioning, balancing of loads on a joint, and size of a permanent prosthesis. Once the stem 107 is attached to femur 201 and the joint cup 205 prepared, the surgeon may attach shim 117 and ball joint 111 to the neck 109. The ball joint 111 may be a pre-determined size that the surgeon has determined may fit the patient. Once the shim 117 and ball joint 111 are attached to the neck 109, the surgeon may insert the ball joint into joint cup 205. The surgeon may then move individual parts of the leg of the patient, for example the femur 201, and observe the force magnitude and position on a GUI, such as GUI 1201 or 1203, on the electronic display 104 of the remote system 103. If the surgeon determines that the ball joint 111 trial insert is the incorrect size, the surgeon may remove the ball joint 111 by applying a finger or tool to the shim 117 release arm 501 and reattach a ball joint 111 of a different size. The surgeon may repeat these steps until a correctly sized ball joint is found. When the surgeon determines that the ball joint 111 trial insert is the correct size, the surgeon may remove the ball joint 111 by using the shim 117 release arm 501. The surgeon may then attach a similarly correctly sized permanent prosthetic ball joint 111 to the neck 109, and install medical device 101 in joint cup 205 as a permanent prosthetic. The disclosed aspects of the present disclosure may be used in any medical device 101 that may need to measure force at a joint. For example, the device of the present disclosure may be a femoral trial head 111 that may be attached to a femoral stem 107 to measure at least load magnitude and location at a hip joint. In other examples, aspects of this disclosure may be incorporated into prosthetic implants or trial inserts for other joints, such as the shoulder joint.

As discussed above, the femoral trial head 111 may include three strain gauge sensors 105 positioned circumferentially around a central circuit board 311 and positioned at an equal distance from the center of column 307, and each sensor 105 is spaced equally from each adjacent sensor 105. The strain gauge sensors 105 may be impacted by sensor impacting features 315 on the interior surface 1103 of the upper housing 115. The sensor impacting features 315 transfer the force on the upper housing 115 to the strain gauge sensors 105 so that the entire force may be measured. The different loads measured by each strain gauge sensor 105 may be processed to determine the load magnitude and location on the femoral trial head 111.

The femoral trial head 111 may measure load magnitude and location for loads within target area 1007, which may be a spherical polygon, on the upper housing 115 of the ball joint 111. Once the strains are measured, the measured strains may be transmitted to the remote system 103 where they are used to calculate the load parameters, such as magnitude and location of the applied load, of the joint in real time. The load parameters are then electronically displayed on a GUI on an electronic display 1307 of the remote system 103. The GUI may be a ring GUI 1201 that displays a ball joint 111 with concentric rings 1209 separating portions of the ball joint 111. The rings 1209 may light up to indicate the distance of the load from the center 102 of the ball joint 111 relative to the stem 107 or neck 109. The color of the lit rings 1209 may change depending on the magnitude of the load. A surgeon may utilize the load location and magnitude. This information may be useful in the relative positioning, orientation, and alignment of the hip and femur. This information may also be useful in the selection of a permanent prosthetic, such as the size of a permanent prosthetic ball joint 111.

It will be apparent to those skilled in the art that various modifications and variations can be made to the disclosed system without departing from the scope of the disclosure. Other embodiments of the system will be apparent to those skilled in the art from consideration of the specification and practice of the joint measurement device disclosed herein. It is intended that the specification and examples be considered as exemplary only, with a true scope of the disclosure being indicated by the following claims and their equivalents.

What is claimed is:

1. A measurement device, comprising:
a stem configured to couple to a bone at a proximal end portion;
a neck extending outward from a distal end portion of the stem;
a ball joint coupled to the neck, the ball joint comprising a lower housing and
an upper housing;
wherein the lower housing comprises:
a center body with a platform at a first end of the center body;
a circuit board with electronic circuitry coupled to the platform; and
a plurality of sensors circumferentially arranged around the center body and spaced from the platform; and
wherein the ball joint is configured to couple to a joint and to measure a load magnitude and location at the joint.

2. The measurement device of claim 1, further comprising:
a shim;
wherein the neck fits inside the shim and the shim fits at least partially inside an opening in the lower housing.

3. The measurement device of claim 1, further comprising at least one battery within the ball joint.

4. The measurement device of claim 1, wherein the sensors include strain gauges.

5. The measurement device of claim 1, wherein the lower housing further comprises sensor holding features configured to hold the sensors at an offset height and an offset angle from the platform.

6. The measurement device of claim 1, further comprising:
lower housing snap features on the lower housing;
upper housing snap features on the upper housing;
wherein the lower housing snap features and the upper housing snap features are both equal in number to the number of sensors;
wherein the lower housing and the upper housing are coupled together with the lower housing snap features and the upper housing snap features; and
wherein the lower housing snap features and the upper housing snap features are aligned with the sensors.

7. The measurement device of claim 1, wherein the sensors are coupled to flexible circuit board portions that are coupled to the circuit board.

8. The measurement device of claim 1, wherein the electronic circuitry is connected to an external antenna positioned outside the ball joint, and wherein the external antenna is configured to communicate with a remote system.

9. The measurement device of claim 1, wherein the measurement device is configured to communicate with a remote system and the remote system displays the load magnitude and location on a graphical user interface.

10. The measurement device of claim 9, wherein the graphical user interface includes a display of the ball joint with concentric rings configured to indicate a distance of the force from the center of the upper housing.

11. A measurement device, comprising:
a stem configured to couple to a bone at a proximal end portion;
a neck extending outward from a distal end portion of the stem;
a shim coupled to the neck;
a ball joint coupled to the shim, the ball joint comprising a lower housing and an upper housing;
wherein the lower housing comprises:
a center body with a platform at a first end of the center body;
a circuit board coupled to the platform; and
three sensors circumferentially arranged around the center body and spaced from the platform; and
wherein the ball joint is configured to couple to a joint and to measure a load magnitude and location at the joint.

12. The measurement device of claim 11, wherein the shim includes release arms that are accessible through gaps in the lower housing.

13. The measurement device of claim 11, further comprising at least one battery within the ball joint positioned on the side of the column.

14. The measurement device of claim 11, further comprising:
lower housing snap features on the lower housing;
upper housing snap features on the upper housing;
wherein the lower housing snap features and the upper housing snap features are both equal in number to the number of sensors;
wherein the lower housing and the upper housing are coupled together with the lower housing snap features and the upper housing snap features; and
wherein the lower housing snap features and the upper housing snap features are aligned with the sensors.

15. The measurement device of claim 11, wherein the measurement device is configured to communicate with a remote system, wherein the remote system is configured to display the load magnitude and location on a graphical user interface, and the graphical user interface includes a display of the ball joint with concentric rings configured to indicate a distance of the force from the center of the upper housing.

16. A measurement device, comprising:
a stem configured to couple to a bone at a proximal end portion;
a neck extending outward from a distal end portion of the stem;
a shim coupled to the neck;
a ball joint coupled to the shim, the ball joint comprising a lower housing and
an upper housing;
wherein the lower housing comprises:
a center body with a platform at a first end of the center body;
a circuit board with electronic circuitry coupled to the platform; and
three sensors circumferentially arranged around the center body;
wherein the three sensors are at an offset height and an offset angle from the platform;
wherein the neck is positioned inside the shim and the shim is positioned at least partially inside an opening in the lower housing; and
wherein the ball joint is configured to couple to a joint and to measure a load magnitude and location at the joint.

17. The measurement device of claim 16, wherein the sensors are coupled to flexible circuit board portions, and wherein the flexible circuit board portions are coupled to the circuit board.

18. The measurement device of claim 17, wherein the lower housing further comprises sensor holding features configured to hold the sensors at the offset height and angle.

19. The measurement device of claim 18, further comprising:
lower housing snap features on the lower housing;
upper housing snap features on the upper housing;
wherein the lower housing snap features and the upper housing snap features are both equal in number to the number of sensors;
wherein the lower housing and the upper housing are coupled together with the lower housing snap features and the upper housing snap features; and
wherein the lower housing snap features and the upper housing snap features are aligned with the sensors.

20. The measurement device of claim 16, wherein the measurement device is configured to communicate with a remote system and the remote system displays the load magnitude and location on a graphical user interface, and the graphical user interface includes a display of the ball joint with concentric rings configured to indicate a distance of the force from the center of the upper housing.
